(19) United States
(12) Patent Application Publication
(10) Pub. No.: US 2007/0038176 A1
(43) Pub. Date: Feb. 15, 2007

(54) Medical devices with machined layers for controlled communications with underlying regions

(76) Inventors: Jan Weber, Maple Grove, MN (US); Thomas J. Holman, Princeton, MN (US)

Correspondence Address: MAYER & WILLIAMS PC, 251 NORTH AVENUE WEST, 2ND FLOOR, WESTFIELD, NJ, 07090, US

(21) Appl. No.: 11/174,821

(22) Filed: Jul. 5, 2005

(51) Int. Cl. A61M 31/00; A61M 37/00

(52) U.S. Cl. 604/93.01

(57) ABSTRACT

According to an aspect of the present invention, implantable or insertable medical devices (also referred to as internal medical devices) are provided. These medical devices include at least one machined layer, at least a portion of which is disposed over at least one underlying region (e.g., a therapeutic agent containing region, a catalytic region, etc.). The at least one machined layer contains a plurality of excavated regions which promote the transport of molecular species across the machined layer. An advantage of the present invention is that medical devices are provided, in which the transport of species into the medical device, out of the medical device, or both are controlled, and may be customized, as desired.

### FIELD OF THE INVENTION

The present invention relates to medical devices which are able to regulate the transport of chemical species between an underlying region of the medical device and an outside environment.

### BACKGROUND OF THE INVENTION

The in vivo delivery of biologically active agents within the body of a patient is common in the practice of modern medicine. In vivo delivery of biologically active agents is often implemented using medical devices that may be temporarily or permanently placed at a target site within the body. These medical devices can be maintained, as required, at their target sites for short or prolonged periods of time, delivering biologically active agents at the target site.

For example, numerous polymer-based medical devices have been developed for the delivery of therapeutic agents to the body. Examples include drug eluting coronary stents, which are commercially available from Boston Scientific Corp. (TAXUS), Johnson & Johnson (CYPHER), and others.

In accordance with certain delivery strategies, a therapeutic agent is provided within or beneath a biostable or bioresorbable polymeric layer that is associated with a medical device. Once the medical device is placed at the desired location within a patient, the therapeutic agent is released from the medical device with a profile that is dependent, for example, upon the loading of the therapeutic agent and upon the nature of the polymeric layer.

Controlling the rate of therapeutic agent release and the overall dose are key parameters for proper treatment in many cases.

### SUMMARY OF THE INVENTION

According to an aspect of the present invention, implantable or insertable medical devices (also referred to herein as internal medical devices) are provided. These medical devices include at least one machined layer, at least a portion of which is disposed over at least one underlying region (e.g., a therapeutic agent containing region, a catalytic region, etc.). The at least one machined layer contains a plurality of excavated regions which promote the transport of molecular species across the machined layer.

An advantage of the present invention is that medical devices are provided, in which the transport of species into the medical device, out of the medical device, or both are controlled, and may be customized, as desired.

The above and many other aspects, embodiments and advantages of the present invention will become clear to those of ordinary skill in the art upon reviewing the detailed description and claims to follow.

### BRIEF DESCRIPTION OF THE DRAWINGS

FIGS. 1A, 1B and 1C are each schematic cross-sectional illustrations of a portion of a medical device surface, in accordance with three aspects of the present invention.

FIGS. 2-7 are each schematic cross-sectional illustrations of a portion of a medical device surface, in accordance with various additional aspects of the present invention.

### DETAILED DESCRIPTION OF THE INVENTION

A more complete understanding of the present invention is available by reference to the following detailed description of numerous aspects and embodiments of the invention. The detailed description of the embodiments which follows is intended to illustrate but not limit the invention.

All publications, patents and patent applications cited herein, whether supra or infra, are hereby incorporated by reference in their entirety.

According to an aspect of the present invention, implantable or insertable medical devices (also referred to herein as internal medical devices) are provided which contain at least one machined layer, which is disposed over at least one underlying region and which includes one or more excavated regions (commonly many more, e.g., 10 to 100 to 1000 to 10,000 to 100,000 to 1,000,000 or more excavated regions).

“Excavated regions” are voids (e.g., holes, slots, etc.) that have been created by the removal of material (i.e., excavation) using techniques with which the fabricator may control the location and shape (i.e., the length, width and depth) of the excavated regions. The excavated regions may be of any size and shape, and may extend partially or completely through the material in which they are formed. Typically, the manufacturing tolerances of the techniques that are used to form the excavated regions are generally tight. For example, where laser radiation is used to form the excavated regions, typical tolerances are on the order of the wavelength of the laser. (Even finer tolerances may be achieved through the use of laser machining techniques whereby laser energy is used indirectly to structure the surface. See, e.g., the reference by Y. Lu and S. C. Chen that is discussed in more detail below.)

Examples of techniques for forming machined layers for use in the invention include direct-write techniques, as well as mask-based techniques in which masking is used to protect portions of the machined layers that are not excavated.

Direct write techniques include those in which excavated regions are created through contact with solid tools (e.g., microdrilling, micromachining, etc., using high precision equipment such as high precision milling machines and lathes) and those that form excavated regions without the need for solid tools (e.g., those based on directed energetic beams such as laser, electron, and ion beams). In the latter cases, techniques based on diffractive optical elements (DOEs), holographic diffraction, and/or polarization trepanning, among other beam manipulation methods, may be employed to generate direct-write patterns as desired. Using these and other techniques many voids can be ablated in a material layer at once.

Mask-based techniques include those in which the masking material contacts the layer to be machined, for example, masks formed using known lithographic techniques, including optical, ultraviolet, deep ultraviolet, electron beam, and x-ray lithography, and those in which the masking material does not contact the layer to be machined, but which is provided between a directed source of excavating energy and the material to be machined (e.g., opaque masks having apertures formed therein, as well as semi-transparent masks such as gray-scale masks which provide variable beam intensity and thus variable machining rates). Material is removed in regions not protected by the above masks using any of a range of processes including physical processes (e.g., thermal sublimation and/or vaporization of the material that is removed), chemical processes (e.g., chemical breakdown and/or reaction of the material that is removed), or a combination of both. Specific examples of removal processes include wet and dry (plasma) etching techniques, and ablation techniques based on directed energetic beams such as laser, electron, and ion beams.

Laser ablation is a technology that uses laser radiation to machine a material of interest. As would be expected, the energy per unit area (fluence) that is required for ablation is material dependent. While likely an oversimplification, two types of ablation mechanisms are commonly discussed: photolytic processes and pyrolytic processes. In pyrolytic processes, the laser energy heats the material, leading to a temperature rise, and subsequent melting, sublimation and/or evaporation of the material. In photolytic processes the photon energy leads to photon-induced chemical reactions, including those that overcome the chemical bonding energy of the molecules in the material to be machined (e.g., polymers may be transformed into smaller, often gaseous, monomers, as well as other molecules and atoms). In certain beneficial embodiments, the excavation process is mostly or completely photolytic in nature. Such processes are sometimes referred to as “cold ablation”.

In certain laser ablation embodiments of the invention, shorter wavelength light is preferred. There are several reasons for this. For example, shorter wavelength light such as UV and deep-UV light can be imaged to a smaller spot size than light of longer wavelengths (e.g., because the minimum feature size is limited by diffraction, which increases with wavelength). Such shorter wavelength light is also typically more photolytic, displaying less thermal influence on surrounding material. Moreover, many materials have high absorption coefficients in the ultraviolet region. This means that the penetration depth is small, with each pulse removing only a thin layer of material, thereby allowing precise control of the drilling depth.

Various lasers are available for laser ablation. For example, excimer lasers are a family of pulsed lasers that are capable of operating in the ultraviolet region of the spectrum. Laser emission is typically generated in these lasers using a gas such as a halogen-based gas (e.g., fluorine, chlorine, hydrogen chloride, etc.) and/or a noble gas (e.g., krypton, argon, xenon, etc.). The particular gas or gas combination employed determines the output wavelength. Available excimer lasers include F₂ (157 nm wavelength), ArF (193 nm), KrCl (222 nm), KrF (248 nm), XeCl (308 nm), and XeF (351 nm) lasers. The average power for these lasers is commonly in the range of 10 W to 1 kW, and the pulse length may be, for example, in the 10-20 ns range. Bulk mass removal, even from fine excavations such as 1 micron holes, has been demonstrated using such lasers.

Solid state lasers include those based on Nd:YAG and Nd:vanadate, among other crystals. Nd:YAG lasers are capable of generating pulse widths of, for example, 10 to 100 ns, and higher harmonic Nd:YAG lasers are capable of generating green 532 nm and UV (355 or 266 nm) beams. Hence, such lasers are capable of operating in the same wavelength and pulse length domains as the excimer lasers. Although the average output of these lasers is one to two orders less than that of excimer lasers, the peak power intensity is high (10⁷-10⁸ W/cm²) because of the short pulse length and high beam quality.

Metal vapor lasers are known, including copper vapor lasers, which generate 510.6 nm (green) and 578.2 nm (yellow) wavelengths with a pulse duration 20-50 ns. The light of copper vapor lasers can also be frequency doubled to 255 nm (second harmonic green) or 289 nm (second harmonic yellow) UV wavelengths. Such lasers are also capable of operating in the same pulse length domain as excimer lasers. Although the pulse energies from these lasers are considerably less than excimer lasers, they have a good spatial coherence and a low divergence, meaning that even with low pulse energy the fluences necessary for machining can readily be provided. Consequently, the removal rates are similar to those obtained by excimer lasers, while the repetition frequency can be much higher. Working with UV copper vapor lasers generally requires relatively expensive UV optics, which are prone to degradation.

A recent generation of pulsed lasers are the so-called femtosecond lasers, which are capable of generating extremely short laser pulses, e.g., 10⁻¹² second to 10⁻¹³ second to 10⁻¹⁴ second to 10⁻¹⁵ second, or even less, commonly between 1 and 1000 fs (1×10⁻¹⁵ second to 1×10⁻¹² second) at present. A specific example of such a system is a chirped pulse amplification (CPA) Ti:sapphire laser, which may generate laser pulses having durations, for example, between 5 and 150 fs (5×10⁻¹⁵ to 1.5×10⁻¹³ second) and may have wavelengths, for example, between 650 and 1100 nm.

There are various advantages of using femtosecond lasers to perform ablation of various materials including polymers. For example, femtosecond lasers allow one to use longer wavelength lasers including infrared lasers for the etching UV-sensitive materials. This is generally believed to be due to the fact that femtosecond laser pulses are so intense that two or more photons can interact simultaneously with electrons in the material to be machined, allowing these lasers to provide energies that are equivalent to that of UV light. Moreover, heat diffusion can be strongly suppressed with such lasers, resulting in high precision and minimal heat influence within the material. In addition, laser energy is transferred to the material so quickly that there is little or no interaction with the resulting plume of vaporized material, which may distort and bend the incoming beam. Furthermore, because the plasma plume is known to leave the surface very rapidly, little or no interaction with the next laser pulse is typically experienced. Finally, since the pulse is very short, atoms in a material to be ablated are believed to be nearly stationary in space with respect to the pulse duration. Consequently, the laser pulse does not react in a significantly different fashion between various types of materials, including dielectric materials and electric materials, allowing essentially any material, including organic and inorganic materials such as polymers, glasses, ceramics, semiconductors, and metallic materials, to be ablated with very high precision, and without damaging surrounding areas as a result of thermal effects.

Further information on laser ablation may be found in Lippert T, and Dickinson J T, “Chemical and spectroscopic aspects of polymer ablation: Special features and novel directions,” Chem. Rev., 103(2): 453-485 February 2003; Meijer J, et al., “Laser Machining by short and ultrashort pulses, state of the art and new opportunities in the age of photons,” Annals of the CIRP, 51(2), 531-550, 2002, and U.S. Pat. No. 6,517,888 to Weber, each of which is hereby incorporated by reference.

Finally, Y. Lu and S. C. Chen, “Micro and nano-fabrication of biodegradable polymers for drug delivery,” Advanced Drug Delivery Reviews 56 (2004) 1621-1633, describe a technique whereby the illumination of a nanometer-sized sphere array using a laser beam is employed to pattern a solid surface in a mass production fashion. More specifically, a 1% (w/v) colloid of silica spheres (diameter=640 nm) was dropped onto a poly(ε-caprolactone) substrate, followed by evaporation under controlled humidity. As the solvent evaporated, capillary forces drew the nanospheres together, and the nanospheres reorganized themselves in a hexagonally close-packed pattern on the substrate (although the as-deposited nanosphere array may, of course, include a variety of defects that arise as a result of nanosphere polydispersity, site randomness, point defects, line defects, etc.). Samples were irradiated with the second and third harmonic wave of an Nd:YAG laser or an ArF excimer laser, yielding nano-hole arrays. Laser energy was varied from a minimum threshold energy, below which no clear nanostructure was observed, to a maximum energy, beyond which the polymer surface was ablated directly by the laser pulse. Perhaps not surprisingly, features were cleaner as the laser wavelength decreased.

Using the above and other techniques, excavated regions of almost any desired shape and depth may be formed. It is noted that, in the final device, the excavated regions need not extend to the exterior surface of the device, but may be formed and then covered by an overlying layer (e.g., a hydrogel layer, among many others). As noted above, the excavated regions may extend completely through the machined layer, or they may extend only partially through the machined layer. For example, the excavated regions may be in the form of numerous orifices which extend completely through the machined layer (e.g., through holes) and provide paths of reduced resistance to transport of various species across the machined layer. As another example, the excavated regions may be in the form of orifices which do not extend completely through the machined layer (e.g., blind holes), but which form thinned regions (a) which may reduce resistance to transport of various species across the machined layer and/or (b) in the event that a bioresorbable material is used to construct the machined layer, which may preferentially degrade over time such that the excavated regions ultimately extend completely through the machined layer. As another example, the excavated region(s) may correspond to a region which has a textured surface.

Shapes for the excavated regions vary widely and include (a) excavations in which the length and width are of similar scale (e.g., holes, including blind holes and through holes) and whose perimeter may be of irregular or regular geometry (e.g., circular, oval, triangular, square, rectangular, pentagonal, etc.), (b) excavations in which the length significantly exceeds the width (e.g., trenches and valleys), which may be, for example, of constant or variable width, and may extend along the surface in a linear fashion or in a nonlinear fashion (e.g., serpentine, zig-zag, etc.), and (c) excavations that are so extensive so as to create protrusions, including protrusions whose length and width are of similar scale and whose perimeters may be regular or irregular (e.g., pillars, domes, knobs, mesas, etc.) and protrusions whose lengths significantly exceed their widths (e.g., ridges), which may be of constant or variable width, which may extend along the surface in a linear or nonlinear fashion, and so forth. Consequently, the cross-sectional area of the excavated region can range from on the order of a square micron or less (e.g., where numerous laser-drilled holes are provided) up to the point where the majority of the device surface is excavated (e.g., wherein substantial portions of the machined layer are excavated to produce protrusions).

Walls that may be created during the formation of the excavated regions include vertical walls, non-vertical walls that result in depressions whose cross-sectional area decreases with increasing depth, non-vertical walls that result in depressions whose cross-sectional area increases with increasing depth, non-vertical walls that result in protrusions whose cross-sectional area decreases with increasing height, non-vertical walls that result in protrusions whose cross-sectional area increases with increasing height, and so forth.

Using the above and other techniques, excavated regions may be formed in layers having of a wide variety of chemical compositions. Materials that may be machined include materials that are biostable and those that are bioresorbable. Materials that may be machined include (a) organic materials (i.e., materials containing 50 wt % or more organic species), such as polymeric materials (i.e., materials containing 50 wt % or more polymers) as well as non-polymeric organic materials (i.e., materials containing 50 wt % or more organic species that are not polymers, for example, non-polymeric organic species such as phospholipids among many others), and (b) inorganic materials (i.e., materials containing 50 wt % or more inorganic species), such as metallic materials (e.g., metals and metal alloys) and non-metallic materials (e.g., including carbon, semiconductors, glasses and ceramics containing various metal- and non-metal-oxides, various metal- and non-metal-nitrides, various metal- and non-metal-carbides, various metal- and non-metal-borides, various metal- and non-metal-phosphates, and various metal- and non-metal-sulfides, among others).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic materials may be selected, for example, from the following: metal alloys such as cobalt-chromium alloys, nickel-titanium alloys (e.g., nitinol), cobalt-chromium-iron alloys (e.g., elgiloy alloys), nickel-chromium alloys (e.g., inconel alloys), and iron-chromium alloys (e.g., stainless steels, which contain at least 50% iron and at least 11.5% chromium), biostable metals such as gold, platinum, palladium, iridium, osmium, rhodium, titanium, tungsten, and ruthenium, and bioresorbable metals such as magnesium.

Specific examples of polymeric and other high molecular weight organic materials may be selected, for example, from materials containing one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF), including elastomeric copolymers of vinylidene fluoride and hexafluoropropylene; silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®g, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

One function of the excavated regions is to improve transport of species across the machined layers of the present invention. For example, such excavated regions may be provided (a) to give species that are outside the medical device improved access to regions that are beneath the machined layers (e.g., to give biological fluids, which may include materials to be catalyzed by or otherwise interact with underlying therapeutic regions, better access to the regions underlying the machined layers) and/or (b) to give species that are beneath the machined layers improved transport to the outside of the medical device (e.g., to improve the ability of species beneath the machined layers, such as therapeutic agents, catalyzed biological products, degradation products, and so forth, to exit the device). Transport of species across the machined layers of the invention may be improved in accordance with the present invention, for example, by increasing the number, surface area and/or depth of the excavated regions. Consequently, the number and/or size of the excavated regions of the invention may be varied to affect transport, as well as other properties, such as cell growth.

For example, it is known that surface roughness can have a significant effect upon cellular attachment. In this regard, the excavated regions may be of a size and shape such that cellular growth at the surface of the medical device is promoted. If desired, one or more growth enhancing agents may be provided on, within, or beneath the machined layer(s).

In other embodiments, cellular growth is not desired. In these embodiments, cell-growth-resistant coatings may be employed. For example, carbon coatings are known to discourage cell attachment. In this regard, it may be possible to create excavated regions (e.g., create laser-drilled holes) that are sufficiently small so as to have a negligible effect on the growth retarding nature of the coating, while at the same time allowing transport of species (e.g., growth retarding species, among others) across the machined layer. As another example, excavated regions may be made only on certain surfaces of the medical device (e.g., to deliver the therapeutic agent into the body), whereas other surfaces are left unexcavated so as to avoid encouraging cell growth.

“Therapeutic agents,” drugs,” “bioactive agents” “pharmaceuticals,” “pharmaceutically active agents”, and other related terms may be used interchangeably herein and include genetic and non-genetic therapeutic agents. Therapeutic agents may be used singly or in combination.

A wide range of therapeutic agent loadings can be used in conjunction with the devices of the present invention, with the pharmaceutically effective amount being readily determined by those of ordinary skill in the art and ultimately depending, for example, the nature of the therapeutic agent itself, the condition being treated, the nature of the machined region(s) within the medical device, and so forth.

Therapeutic agents may be selected, for example, from the following: adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, anti-amebic agents, anti-androgen agents, anti-anemic agents, anti-anginal agents, anti-anxiety agents, anti-arthritic agents, anti-asthmatic agents, anti-atherosclerotic agents, antibacterial agents, anticholelithic agents, anticholelithogenic agents, anticholinergic agents, anticoagulants, anticoccidal agents, anticonvulsants, antidepressants, antidiabetic agents, antidiuretics, antidotes, antidyskinetics agents, anti-emetic agents, anti-epileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipoproteinemic agents, antihypertensives, antihypotensives, anti-infective agents, anti-inflammatory agents, antikeratinizing agents, antimicrobial agents, antimigraine agents, antimitotic agents, antimycotic agents, antineoplastic agents, anti-cancer supplementary potentiating agents, antineutropenic agents, antiobsessional agents, antiparasitic agents, antiparkinsonian drugs, antipneumocystic agents, antiproliferative agents, antiprostatic hypertrophy drugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, cardioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMGCoA reductase inhibitors, immunizing agents, immunomodulators, immunoregulators, immune response modifiers, immunostimulants, immunosuppressants, impotence therapy adjuncts, keratolytic agents, LHRH agonists, luteolysin agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine Al antagonists, serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like.

Numerous additional therapeutic agents useful for the practice of the present invention may be selected from those described in paragraphs [0040] to [0046] of commonly assigned U.S. Patent Application Pub. No. 2003/0236514, the disclosure of which is hereby incorporated by reference. Examples include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasoditating agents, and agents that interfere with endogenous vasoactive mechanisms, among others.

Some specific beneficial therapeutic agents include vascular endothelial growth factors (e.g., VEGF-2), antithrombotic agents (e.g., heparin), antirestenotic agents such as paclitaxel (including particulate forms thereof such as ABRAXANE albumin-bound paclitaxel nanoparticles), sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, and Serca 2 gene/protein, resiquimod, imiquimod (as well as other imidazoquinoline immune response modifiers), human apolioproteins (e.g., AI, AII, AIII, AIV, AV, etc.), as well a derivatives of the forgoing, among many others.

Examples further include polymeric and non-polymeric entities, which contain ion-exchange or chelating functional groups that selectively bind calcium and can be used for calcium removal. Functional groups such as imino diacetic acid groups and —CH₂—NH—CH₂—PO₃⁻ groups (e.g., —CH₂—NH—CH₂—PO₃Na) are used in commercially available microporous resin products for calcium extraction/removal. Examples include lonac SR-5 available from Sybron Chemicals, Inc., Birmingham, N.J., USA, and Amberlite IRC 747 available from Available from Rohm and Haas Australia, Camberwell, Victoria 3124, Australia. Ethylene diamine tetra acetic acid (EDTA) and various other amino acid options have been demonstrated as efficient chelating agents for calcium.

In those embodiments where one or more therapeutic agents are provided, they may be, for example, disposed on, within and/or beneath the machined layers of the invention. For example, one or more therapeutic agents may be (a) provided within a region that overlies a machined layer, (b) provided at a surface of (e.g., covalently or non-covalently attached to) a machined layer, (c) provided within a machined layer, (d) provided at a surface of (e.g., covalently or non-covalently attached to) a region that underlies a machined layer, (e) provided within a region that underlies a machined layer, and so on.

Where one or more therapeutic agents are provided within a region that underlies a machined layer, the therapeutic agent containing region may be, for instance, a therapeutic agent containing layer that is disposed over an underlying substrate, in which case the therapeutic agent containing layer may be applied with or without some form of matrix, such as a polymeric matrix. The substrate may be formed from a variety of materials including organic and inorganic materials such as those described above.

If desired a plurality of therapeutic-agent containing layers may be provided, for example, disposed laterally with respect to one another or stacked on top of one another.

As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

Excavated region placement may be engineered for various clinical rationales. For instance, in the case of a stent, excavated regions may be provided only on the surface of the stent facing the vessel wall, so as to minimize systemic effects. The number and/or size of the excavated regions may also be varied along the length of the device. For example, where a stent is utilized, the number of holes at the ends of the stent and/or the size of the holes at the ends of the stent can be varied so as to deliver more or less drug to the ends of the device. In the case of a bifurcation stent, one side or a specific region of the stent may be engineered to deliver more or less drug. Clearly, the variants are endless.

In the case of a blood-contacting vascular device (e.g., a vascular stent), machined layers may be provided on tissue contacting surfaces of the device (e.g., on the exterior surface of a stent) so as to facilitate release of an anti-restenosis agents such as those above and/or anti-inflammatory agents such as N-monomethyl-arginine (e.g., L-NMMA, an inhibitor of the nitric oxide synthase enzyme, NOS, as produced by endothelial cells, converting the amino acid L-arginine to L-citrulline and forming nitric oxide in the process) into the surrounding tissue, as well as on blood contacting surfaces of the device (e.g., the inner and lateral surfaces of a stent) so as to facilitate release of an antithrombogenic drug, obstruction-clearing drug (e.g., where a stent is adapted to be placed upstream of an occlusion, such as a chronic total occlusion) and/or a vasodilator drug such as nitroglycerine.

In some embodiments, an underlying substrate is provided which is bioresorbable, in which case the machined layer can be engineered to regulate the rate of bioresorption of the substrate. In certain of these embodiments, number and/or size of the voids may vary with surface location so as to cause certain portions of the underlying substrate to be bioresorbed more quickly than other portions. Specific examples of medical devices that may be provided with machined layers for bioresorption regulation include those described in U.S. Pat. Pub. No. 2001/0044651 to Steinke et al., in which bioresorbable stents are described which are formed from at least one series of sliding and locking radial elements and at least one ratcheting mechanism comprising an articulating element and a plurality of stops. The ratcheting mechanism permits one-way sliding of the radial elements from a collapsed diameter to an expanded diameter, but inhibits radial recoil from the expanded diameter. For example, the number and/or size of the excavated regions may vary along the length of the stent such that the bioresorption process starts at one end of the stent and works along the length of the stent to the other end. Consequently, the stent disappears much like a burning candle. As a result, the chances are improved that the remaining stent will stay in one piece at all times, rather than falling into several pieces.

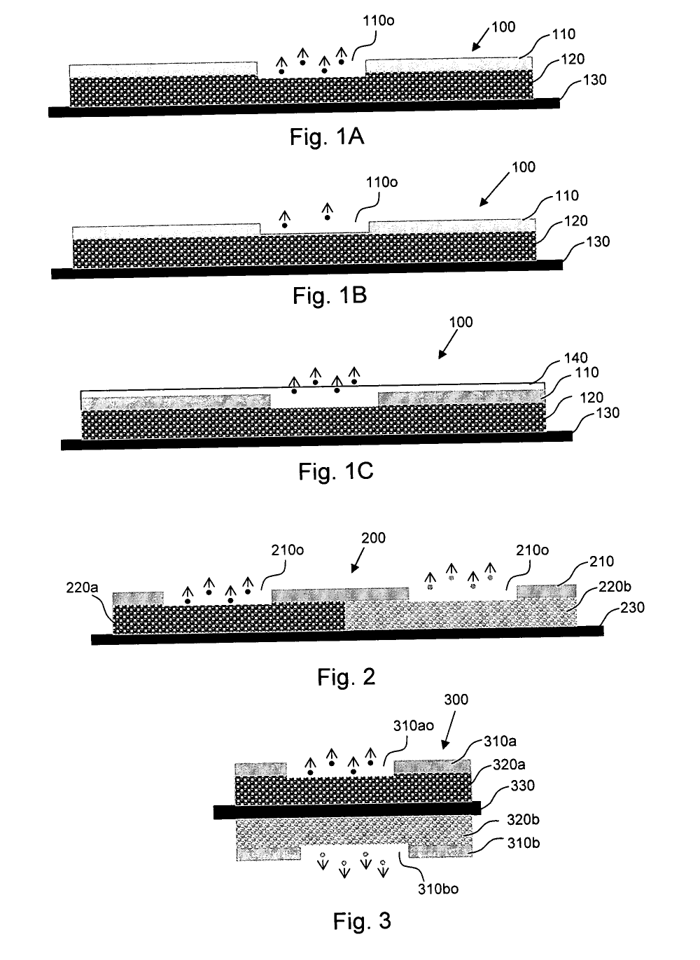

Further embodiments of the invention will now be described with reference to the drawings. Turning now to FIG. 1A, a portion of a medical device 100 (e.g., a stent) is schematically illustrated in cross-section, in which a machined layer 110 having an evacuated region 110o (e.g., a laser drilled hole) is provided over a therapeutic agent containing region 120 (e.g., a polymer matrix containing a therapeutic agent), which is in turn disposed over a medical device substrate 130 (e.g., a stent strut). The evacuated region 110o provides a path of reduced resistance to transport of therapeutic agent (illustrated with dots) across the machined layer 110 and out of the device 100. This combination of layers allows for predetermined kinetic drug release (KDR) over time. By specifying and controlling the composition and thickness of the layers 110, 120 and by controlling the size and number of the evacuated region 110o, the performance of the KDR and the clinical outcome may be assured.

A device 100 like that of FIG. 1A may be formed using the following steps: (a) a drug holding polymer layer 120 is deposited on substrate 130, (b) the amount of deposited drug is measured, for example, by measuring the thickness of the layer 120 using white light interferometry and then inferring the amount of drug based on the result, (c) a barrier layer is deposited over the layer 120, and (d) laser ablation is used to drill holes through the barrier layer, thereby forming the machined layer 110. The number and/or size of the holes may vary, as required, to compensate for any inaccuracy in the amount of deposited drug. (The amount of drug deposited on the substrate may be variable to some extent, e.g., due to limitations in deposition technology, with overall effect being a variation in the drug release profile if compensating actions are not taken.)

FIG. 1B is a device like FIG. 1A, except that the evacuated region 110o does not extend entirely through the machined layer 110. However, resistance to transport of the therapeutic agent across the machined layer 110 is reduced at the evacuated region 110o.

FIG. 1C is a device like FIG. 1A, except that an additional layer 140, such as a hydrogel layer, is provided over the machined layer 110. The material for the additional layer 140 is selected such that it provides negligible resistance to transport of the therapeutic agent from the device 100, relative to the material selected for the layer 110.

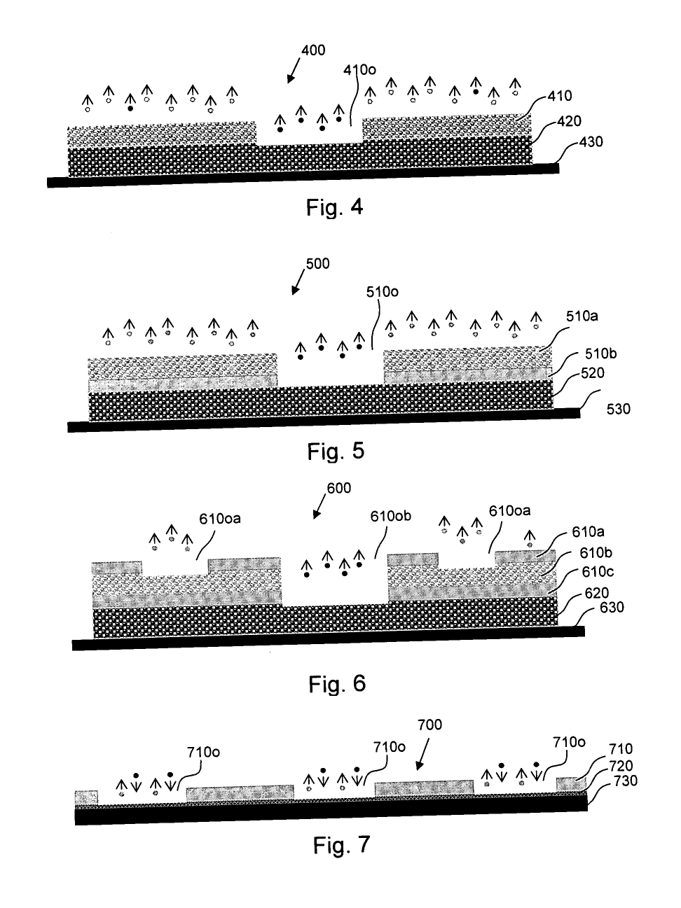

FIG. 2 schematically illustrates a portion of a medical device 200, in which a machined layer 210 having evacuated regions 210o is provided over a pair of therapeutic agent containing regions 220a, 220b, which are in turn disposed over a medical device substrate 230. The therapeutic agent containing regions 220a, 220b differ in composition in FIG. 2 because they contain different drugs. (Of course, such layers could also contain the same drug at different concentrations, contain the same drug with different matrix materials, and so forth.) The evacuated regions 210o provide paths of reduced resistance to transport of the therapeutic agent (again, illustrated with dots) across the machined layer 210 and out of the device 200.

FIG. 3 schematically illustrates a portion of a medical device 300, in which a first machined layer 310a having an evacuated region 310ao is provided over a first therapeutic agent containing region 320a, which is in turn disposed over a medical device substrate 330. The device 300 also contains a second machined layer 310b having a evacuated region 310bo, provided over a second therapeutic agent containing region 320b, which is in turn disposed over a side of the medical device substrate 330 that is opposite from the first therapeutic agent containing region 320a. As in FIG. 2, the therapeutic agent containing regions 320a, 320b differ in composition, because they contain different drugs (although they could also contain the same drug at different concentrations, contain the same drug with different matrix materials, and so forth). The evacuated regions 310ao, 310bo provide paths of reduced resistance to transport of the therapeutic agents (illustrated with dots) across the machined layers 310a, 310b and out of the device 300.

As a specific example, region 320a may contain an anti-restenosis drug and, along with machined layer 310a, may be disposed at an outer surface of a stent substrate 300, while region 320b may contain an anti-thrombotic drug and, along with machined layer 310b, may be disposed at an inner surface of the stent substrate 300.

FIG. 4 schematically illustrates a portion of a medical device 400 (e.g., a stent), in which a therapeutic agent containing machined layer 410, which contains an evacuated region 410o, is provided over a therapeutic agent containing region 420, which is in turn disposed over a medical device substrate 430. As above the therapeutic agents within machined layer 410 and region 420 may be the same (e.g., at different concentrations) or they may be different (as illustrated, they are different). In FIG. 4, the machined layer 410 acts as a therapeutic agent releasing layer, and it also acts to regulate the transport of the therapeutic agent in the therapeutic agent containing region 420 lying beneath it. Note that FIG. 4 is analogous to FIG. 1A, except that the machined layer 410 in FIG. 4 contains a therapeutic agent, whereas the machined layer 110 in FIG. 1A does not.

FIG. 5 schematically illustrates a portion of a medical device 500 (e.g., a stent) having a first machined layer 510a, which contains a therapeutic agent, and a second machined layer 510b, which does not. An evacuated region 510o extends through the first and second machined layers 510a, 510b. First and second machined layers 510a, 510b are disposed over a therapeutic agent containing region 520, which is in turn disposed over a medical device substrate 530. As above, the therapeutic agents within the first machined layer 510a and the therapeutic agent containing region 520 may be different or they may be the same (as illustrated, they are different). In this embodiment, the first machined layer 510a acts as a therapeutic agent releasing layer, whereas the second machined layer 510b acts to regulate the transport of the therapeutic agent within the therapeutic agent containing region 520.

FIG. 6 schematically illustrates a portion of a medical device 600 having a second machined layer 610b, which contains a therapeutic agent, and first and third machined layers 610a and 610c, which do not. Evacuated regions 610oa extend through the first machined layer 610a, whereas evacuated region 610ob extends through the first, second and third machined layers 610a, 610b, 610c. First, second and third machined layers 610a, 610b, 610c, are disposed over a therapeutic agent containing region 620, which is in turn disposed over a medical device substrate 630. As above, the therapeutic agents within the second machined layer 610b and the therapeutic agent containing region 620 may be the same or different (as illustrated, they are different). In this embodiment, the first machined layer 610a acts to regulate the transport of the therapeutic agent from the second machined layer 610b, whereas the third machined layer 610c acts to regulate the transport of the therapeutic agent from the therapeutic agent region 620.

In some aspects of the invention, the therapeutic region does not release a therapeutic agent, but rather has an effect upon species that exist outside the medical device. For instance, as noted above, the therapeutic region underlying the machined layer may have a catalytic effect upon species that are present in surrounding biological fluid, for example, resulting in the removal of harmful species, resulting in the production of beneficial species, and so forth. As another example, the therapeutic region underlying the machined layer may act to trap harmful species are present in surrounding biological fluid.

An embodiment of the invention is shown in FIG. 7, which schematically illustrates a medical device 700 that includes a machined layer 710 having evacuated regions 710o. The machined layer 710 is provided over a therapeutic region 720, such as a catalytic region (e.g., a catalytic metal or metal oxide layer, such as a platinum or iridium oxide layer, which is capable of acting as a peroxidase to eliminate potentially harmful peroxide compounds in the environment surrounding the device), which is in turn disposed over a medical device substrate 730. The evacuated regions 710o provide (a) paths of reduced resistance to transport of species from the surrounding environment (e.g., peroxide species, illustrated by black dots) to the surface of the catalytic layer 720, and (b) paths of reduced resistance to transport of catalytically converted species (illustrated by grey dots) from the surface of the catalytic layer 720 into the surrounding environment.

While the medical device in conjunction with the drawings is sometimes referred to as a stent, the present invention is clearly applicable to a wide array of medical devices including a wide array of implantable or insertable medical devices and portions thereof, for example, catheters (e.g., renal or vascular catheters), balloons, catheter shafts, guide wires, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, vascular valves, biopsy devices, patches for delivery of therapeutic agent to intact skin and broken skin (including wounds); tissue engineering scaffolds for cartilage, bone, skin and other in vivo tissue regeneration, as well as a variety of other substrates (which can comprise, for example, glass, metal, polymer, ceramic and combinations thereof) that are implanted or inserted into the body.

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. An implantable or insertable medical device comprising a machined layer at least a portion of which is disposed over an underlying region, said machined layer comprising a plurality of excavated regions which promote transport of molecular species across said machined layer.

2. The medical device of claim 1, wherein said machined layer is a polymeric layer.

3. The medical device of claim 1, wherein said machined layer is a non-polymeric layer.

4. The medical device of claim 1, wherein said machined layer is selected from a metallic layer, a ceramic layer and a carbon layer.

5. The medical device of claim 1, wherein said machined layer is a biostable layer.

6. The medical device of claim 1, wherein said machined layer is a bioresorbable layer.

7. The medical device of claim 1, wherein said medical device comprises a plurality of said machined layers.

8. The medical device of claim 7, wherein at least one machined layer at least partially overlies another machined layer.

9. The medical device of claim 7, wherein at least one machined layer at least partially overlies another machined layer that comprises a therapeutic agent.

10. The medical device of claim 1, comprising a first machined layer provided on a solid-tissue contacting region of said medical device and a second machined layer provided on a fluid contacting region of said medical device.

11. The medical device of claim 1, wherein the machined layer comprises a plurality of excavated regions that extend through the machined layer.

12. The medical device of claim 1, wherein the machined layer comprises a plurality of excavated regions that extend at least halfway, but not completely through, said machined layer.

13. The medical device of claim 1, wherein said excavated regions are laser excavated regions.

14. The medical device of claim 13, wherein said laser excavated regions comprise laser drilled holes, laser drilled trenches, or a combination of both.

15. The medical device of claim 1, wherein said machined layer is at least partially covered by an additional layer.

16. The medical device of claim 15, wherein said additional layer is a hydrogel layer.

17. The medical device of claim 1, wherein said medical device comprises a therapeutic agent.

18. The medical device of claim 17, wherein said therapeutic agent selected from anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

19. The medical device of claim 1, wherein said machined layer comprises a therapeutic agent.

20. The medical device of claim 1, wherein said underlying region comprises a therapeutic agent.

21. The medical device of claim 1, wherein said underlying region is a polymeric layer that comprises a therapeutic agent.

22. The medical device of claim 1, wherein said machined layer comprises a first therapeutic agent, wherein said underlying layer comprises a second therapeutic agent which may be the same as or different from the first therapeutic agent.

23. The medical device of claim 1, wherein said device comprises a plurality of underlying regions.

24. The medical device of claim 1, wherein said device comprises a first underlying region that comprises a first therapeutic agent, and a second underlying region that comprises a second therapeutic agent which may be the same as or different from the first therapeutic agent.

25. The medical device of claim 24, wherein the first underlying region at least partially covers the second underlying region.

26. The medical device of claim 24, wherein neither the first nor the second underlying region at least partially covers the other underlying region.

27. The medical device of claim 1, wherein said device comprises a first machined layer at least partially disposed over a first underlying region that comprises a first therapeutic agent, and a second machined layer at least partially disposed over a second underlying region that comprises a second therapeutic agent which may be the same as or different from the first therapeutic agent.

28. The medical device of claim 27, wherein said first and second therapeutic agents are different.

29. The medical device of claim 27, wherein said first machined layer is at least partially disposed over said first underlying region, wherein said first underlying region is at least partially disposed over said second machined layer, and wherein said second machined layer is at least partially disposed over said second underlying region.

30. The medical device of claim 27, wherein said first machined layer and said first underlying region are disposed over a first portion of a substrate, and said second machined layer and said second underlying region are disposed over a second portion of said substrate.

31. The medical device of claim 30, wherein said first and second portions are located on opposites sides of said substrate.

32. The medical device of claim 1, wherein said underlying region is disposed over an underlying substrate.

33. The medical device of claim 32, wherein said underlying substrate is selected from a metal and a metal alloy substrate.

34. The medical device of claim 1, wherein said underlying region is a catalytic region.

35. The medical device of claim 34, wherein said catalytic region is a metal or metal oxide region.

36. The medical device of claim 1, wherein the underlying region is biostable.

37. The medical device of claim 1, wherein the underlying region is bioresorbable.
